(19) United States
(12) Patent Application Publication
(10) Pub. No.: US 2019/0090813 A1
(43) Pub. Date: Mar. 28, 2019

(54) Patient Restraining Medical Device

(71) Applicant: Jonathan Nover, Brooklyn, NY (US)

(72) Inventor: Jonathan Nover, Brooklyn, NY (US)

(21) Appl. No.: 15/716,119

(22) Filed: Sep. 26, 2017

(51) Int. Cl. A61B 5/00; A61B 5/0205; A61B 5/1455

(52) U.S. Cl. CPC A61B 5/6831; A61B 5/14552; A61B 5/0205; A61B 5/6824

(57) ABSTRACT

A device, system and method for restraining a patient in need thereof, documenting that appropriate and continuous health care is being provided to the restrained patient, and monitoring a vital sign of the patient comprising a wrist band adapted for restraining movement of a patient's wrist from a fixed position, wherein the wrist band is provided with a sensor adapted to monitor a patient's vital sign and a bar code which, upon scanning with a barcode reader, identifies the patient and the time of scanning.

### BACKGROUND OF INVENTION

### 1. Field of the Invention

The invention relates to a system, device and method for restraining and administering health care to a patient.

### 2. Discussion of the Prior Art

Limb restraining devices have been utilized for many years to substantially immobilize one or more limbs of a patient in need thereof, particularly those who have recently suffered trauma, by securing such limbs to a fixed object. For example, patients being transported to a hospital in an emergency vehicle such as, for example, an ambulance on an emergency cot pose a complex problem with many unique and unresolved issues. When transporting patients with such acute medical problems that require constant monitoring, a current practice is to restrain the patient to prevent movement which may exacerbate the patient's medical condition, and to prevent a patient from self-harm or harm to others.

Such devices typically include a cuff member, which can be secured about a limb of a patient to be restrained, and one or more straps. The straps ensure that the restraining device is tight enough so as to substantially preclude the restrained limb from slipping out of securement from the cuff member. Substantial immobilization of the limb to be restrained is then achieved by merely securing one or both ends of the straps to the fixed object. An example of one such limb restraining device is disclosed in U.S. Pat. No. 4,628,925.

Regulatory and safety requirements, however, dictate that health care personnel must continuously assess various biological parameters of the restrained patients.

Additionally, attaching external devices to perform Vital Sign Checks can be very difficult and unsafe when a patient is in a violent crisis.

It is an object of the invention to provide a system, method and device that simultaneously restrains a patient, ensures that the patient is continuously being assessed and enables the monitoring of one or more vital signs of the patient.

### SUMMARY OF THE INVENTION

The invention relates to a method, system, and device for restraining a patient in need thereof, documenting that appropriate and continuous health care is being provided to the restrained patient, and monitoring a vital sign of the patient. More particularly, the invention relates to a wrist band adapted for restraining movement of a patient's wrist from a fixed position, wherein the wrist band is provided with a sensor adapted to monitor a patient's vital sign and a bar code which, upon scanning with a barcode reader, identifies the patient and the time of scanning.

### DESCRIPTION OF THE DRAWINGS

FIG. 1 is a top elevational view of a restraining device of the invention.

FIG. 2 is a bottom elevational view of a restraining device of the invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention is predicated on the unexpected discovery that a patient may be restrained, while ensuring that he/she is being appropriately assessed by monitoring at least one vital sign of the patient utilizing a single unitary device.

More particularly, the invention enables improved standards of care for a patient requiring restraints by providing health care personnel with means to both monitor a vital sign of the patient and document that the assessment was completed, while simultaneously restraining the patient.

A preferred embodiment of the invention comprises a system for restraining a patient in need thereof and documenting that appropriate health care is given to the restrained patient comprising a wrist band adapted for restraining movement of the patients wrist from a fixed position, wherein the wrist band is provided with (1) a pulse oximeter sensor adapted for adhesive fixation to the patients anterior wrist over the ulnar and radial arteries to determine the patient's blood oxygen saturation and (2) a bar code on the opposite side of the wrist band which identifies the patient. Bar code readers are well known in the art and are available off the shelf in a form that can be easily modified and adapted to the present invention.

The system may also include an element for scanning the barcode and documenting the time of scanning and an element for storing the times of scanning and the measured blood oxygen saturation levels.

The method, system and device of the invention will enable the health care giver to spend more time concentrating on the assessment of the restrained patient and less time concerned about administrative paper work, such as recording the times of patient assessment. The act of scanning the restraint demonstrates that the care giver presented at a time certain to the restrained patient to perform the assessment. The adhesive infrared strap of the invention enables continuous monitoring of O₂ saturation and pulse without having to manually attach further external equipment, while keeping the patient and care giver safe. The adhesive strap is attached to the patient's anterior wrist, utilizing the ulnar and radial arteries anatomically present to measure oxygen saturation levels and pulse.

The system of the invention may also include means for scanning the barcode, reading the blood oxygen saturation readings and storing both.

Embodiments of the present invention will be described in detail hereinafter. The examples of these embodiments have been illustrated in the drawings throughout which same or similar reference numerals refer to same or similar elements or elements having same or similar functions. The embodiments described with reference to the drawings are illustrative, merely used for explaining the present invention and should not be regarded as any limitations thereto.

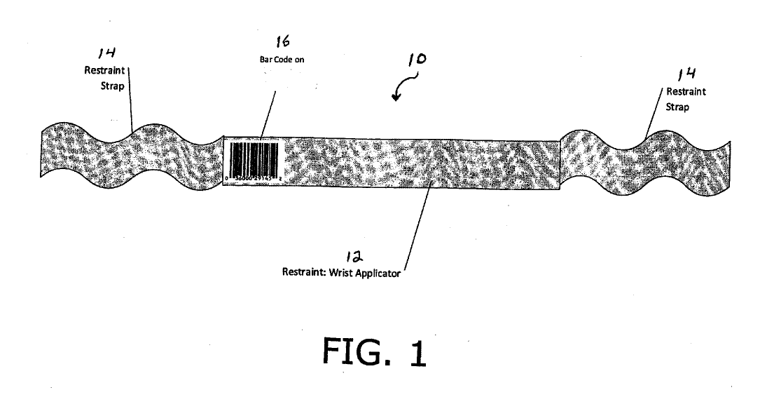

FIG. 1 depicts the exterior side of the restraint device 10 which is exposed when positioned on the patient's wrist. The wrist applicator 12 is provided with straps 14 which are adapted to restrain the patient by affixing them to a fixed object. Positioned on the exterior side of the device is bar code 16 which, when read by a barcode reader (not shown) identifies the patient and the time of each reading.

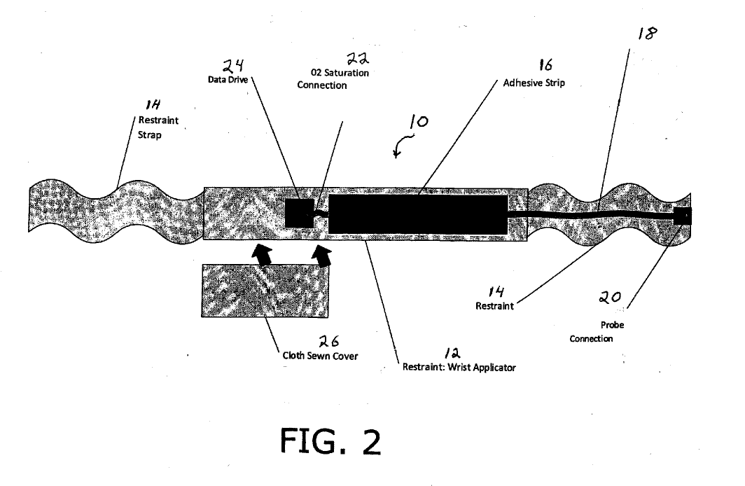

FIG. 2 depicts the interior side of the restraint device 10 which is positioned on the patient's wrist such that the adhesive infrared oximeter strip 16 adheres to the anterior side of the patient's wrist (not shown). The infrared adhesive strip 16 communicates with a data drive 24 via 02 saturation cord 22. Optionally, a cloth cover 26 may be positioned so as to prevent contact between the patient and the data drive 24. The adhesive infrared strip also communicates with pronged probe 20 via cord 18. The probe 20 is adapted for connection to a vital sign/cardiac monitor.

Although various specific constructions and steps have been shown and discussed, these are for illustrative purposes only. Various modifications will be apparent to those of skill in the art. Therefore, the scope of the present invention should be determined with reference to the claims appended hereto.

What is claimed is:

1. A device for restraining a patient in need thereof, documenting that appropriate and continuous health care is being provided to the restrained patient, and monitoring a vital sign of the patient comprising a wrist band adapted for restraining movement of a patient's wrist from a fixed position, wherein the wrist band is provided with a sensor adapted to monitor a patient's vital sign and a bar code which, upon scanning with a barcode reader, identifies the patient and the time of scanning.

2. The device of claim 1 wherein said pulse oximeter sensor is an infrared sensor.

3. The device of claim 1 wherein said sensor is positioned on the wrist band for adhesive fixation to the patient's anterior wrist over the ulnar and radial arteries to determine the patient's blood oxygen saturation.

4. The device of claim 1 wherein the bar code is positioned on the opposite side of the wrist band.

5. The device of claim 1 further comprising elements for association with a processor-implemented system programmed in a non-transitory processor-readable medium for storage of the information provided thereby.

6. A system for restraining a patient in need thereof, documenting that appropriate health care is given to the restrained patient and monitoring a vital sign of the patient comprising (1) a wrist band adapted for restraining movement of the patients wrist from a fixed position, wherein the wrist band is provided with (a) a pulse oximeter sensor adapted for adhesive fixation to the patients anterior wrist over the ulnar and radial arteries and to determine the patient's blood oxygen saturation and (b) a bar code positioned on the opposite side of the band, and elements for reading the barcode and blood oxygen saturation.

7. The system of claim 3 wherein said pulse oximeter sensor is an infrared sensor.

8. The system of claim 3 wherein said bar code and sensor are associated with a processor-implemented system programmed in a non-transitory processor-readable medium for storage of the information provided thereby.

9. A method for restraining a patient in need thereof, documenting that appropriate and continuous health care is being provided to the restrained patient, and monitoring a vital sign of the patient comprising affixing a wrist band to said patient which is adapted for restraining movement of the patient's wrist from a fixed position, wherein the wrist band is provided with a sensor adapted to monitor a patient's vital sign and a bar code which, upon scanning with a barcode reader, identifies the patient and the time of scanning.

10. The method of claim 8, wherein the wrist band is provided with (a) a pulse oximeter sensor adapted for adhesive fixation to the patient's anterior wrist over the ulnar and radial arteries and to determine the patient's blood oxygen saturation and (b) a bar code positioned on the opposite side of the band.

11. The method of claim 9 wherein said bar code and sensor are associated with a processor-implemented system programmed in a non-transitory processor-readable medium for storage of the information provided thereby.
